(19) United States
(12) Patent Application Publication
(10) Pub. No.: US 2010/0152706 A1
(43) Pub. Date: Jun. 17, 2010

(54) ATRAUMATIC SUCTION CATHETER

(76) Inventors: Cassandra E. Morris, Roswell, GA (US); John Brewer, Marietta, GA (US); Emily Reichart, Cincinnati, OH (US)

Correspondence Address: KIMBERLY-CLARK WORLDWIDE, INC.;Tara Pohlkotte, 2300 Winchester Rd., NEENAH, WI, 54956, US

(21) Appl. No.: 12/335,508

(22) Filed: Dec. 15, 2008

(51) Int. Cl. A61M 1/00

(52) U.S. Cl. 604/523

(57) ABSTRACT

An atraumatic suction catheter having a tubular body with a lumen formed therethrough. At a distal end, the catheter has three equally spaced elongated openings positioned near its distal tip. The catheter may have a fourth round opening positioned proximally to the three openings. The catheter has improved suctioning ability of viscous secretions, and reduced impact compared to other suction catheter embodiments.

### BACKGROUND

The invention(s) disclosed herein relate generally to improved medical care for intubated patients, and more particularly to a novel suction catheter for aspiration of mucous and other fluids and secretions from at least a portion of a patient's respiratory tract, namely, the tracheobronchial passages. More particularly, the invention(s) disclosed here relate to suction catheters having improved tip structures which allow for more efficient suctioning of the tracheobronchial passages, while reducing the likelihood of trauma thereto during the suctioning procedure.

Traditionally, suction catheters have consisted of a flexible plastic tube having a lumen formed therethrough. Such suction catheters usually have a beveled distal end or tip with an opening formed in the end which is in axial alignment with the lumen of the catheter. A proximal end of the catheter is configured to connect to a suction vacuum.

Additional openings may be provided adjacent to the distal end of a suction catheter to increase its suctioning capability. These suction catheters, however, have continued to present problems.

When few openings are provided adjacent the distal end, these openings can easily become clogged when high viscosity secretions are suctioned. Therefore, the suction is increased at the larger opening in the distal tip. The increased suction at the distal tip opening can result in trauma to the delicate tissue of the tracheobronchial passages when the tissue of these passages is pulled against the tip during suctioning.

Therefore, suction catheters with a number of openings near the distal tip have been provided, to alleviate this problem. However, in this instance, the plurality of openings may act more like strainers, resulting in multiple blockages, resulting in the same result previously noted. Alternatively, however, a large number of openings near the distal tip of the catheter weakens the structure of near the distal tip. This results in the catheter tip buckling and folding over on itself, such that suctioning is again ineffective or impossible to perform, due the structural failure of the tip of the suction catheter.

Furthermore, when a suction catheter is stiff and has only a few openings at or near the distal tip, the suction catheter may cause impact injury to the delicate tracheobronchial tissue upon insertion against such tissue. Therefore, these catheters can be advanced only with great caution by the health care provider, and may be ineffective at suctioning due less insertion into the respiratory tract of an intubated patient. Even with suction catheters formed of more flexible materials, there is concern among health care providers about catheter insertion injuries which may occur within the respiratory tract of a patient.

There is a need for a suction catheter which effectively suctions both lower and higher highly viscosity secretions and which does not become easily blocked by such secretions. There is a need for a suction catheter which has a sufficient number of openings in and around the distal tip of the suction catheter which do not become blocked and which do not compromise the structure of the catheter. Further, there is a need for a distal tip of a suction catheter which greatly reduces impact injuries against the delicate tracheobronchial tissue when suctioning.

### SUMMARY OF THE INVENTION

In response to the difficulties and problems discussed herein, an atraumatic suction catheter is provided. The suction catheter includes a tube-shaped body having a lumen formed therethrough, and a beveled distal tip having an opening therein in communication with the lumen. A proximal end of the body has an opening in communication with the lumen and adapted to be coupled to a suction source. The catheter also includes three equally-spaced apertures positioned near the distal tip. Each of the three apertures has a perimeter defining a pair of parallel elongated sides and opposing U-shaped ends. The catheter also has one round aperture spaced a distance proximally from the three apertures.

In another aspect of the invention, a suction catheter is provided. The suction catheter includes a tube-shaped body having a lumen formed therethrough, and a beveled distal tip having an opening therein in communication with the lumen. A proximal end of the body has an opening in communication with the lumen and adapted to be coupled to a suction source. The catheter also includes three equally-spaced apertures positioned near the distal tip. Each of the three apertures has a perimeter defining a pair of parallel elongated sides and opposing U-shaped ends. When the distal tip of the suction catheter is moved at a speed of about 0.4 inches/second, it has an impact force of less than about 0.6 lbf.

In yet another aspect of the invention, a suction catheter is provided. The suction catheter includes a tube-shaped body having a lumen formed therethrough, and a beveled distal tip having an opening therein in communication with the lumen. A proximal end of the body has an opening in communication with the lumen and adapted to be coupled to a suction source. The catheter also includes three equally-spaced apertures positioned near the distal tip. Each of the three apertures has a perimeter defining a pair of parallel elongated sides and opposing U-shaped ends. When the distal tip of the suction catheter is moved at a speed of about 0.4 inches/second, the distal tip has an impact ratio less than 1.

### Definitions

As used herein the following terms have the specified meanings, unless the context demands a different meaning, or a different meaning is expressed; also, the singular generally includes the plural, and the plural generally includes the singular unless otherwise indicated.

As used herein, the terms “comprise,” “comprises,” “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof. Similarly, the terms “include”, “includes”, “including,” as well as the terms “has”, “have”, “having” and derivatives thereof, are intended to be interpreted as the word “comprise”, and are intended to be open-ended terms that specify the presence of any stated features, elements, integers, steps, or components, but do not preclude the presence or addition of one or more other features, elements, integers, steps, components, or groups thereof.

As used herein, the term “couple” includes, but is not limited to, joining, connecting, fastening, linking, tying, adhering (via an adhesive), or associating two things integrally or interstitially together.

As used herein, the term “configure” or “configuration”, and derivatives thereof means to design, arrange, set up, or shape with a view to specific applications or uses. For example: a military vehicle that was configured for rough terrain; configured the computer by setting the system's parameters.

As used herein, the terms “substantial” or “substantially” refer to something which is done to a great extent or degree; a significant or great amount; for example, as used herein “substantially” as applied to “substantially” covered means that a thing is at least 70% covered.

As used herein, the term “unitary” refers to a unitary component, i.e., a whole, un-divided, un-separated component formed from one piece of material(s).

As used herein, the term “about” adjacent to a stated number refers to an amount that is plus or minus ten (10) percent of the stated number.

These terms may be defined with additional language in the remaining portions of the specification.

### BRIEF DESCRIPTION OF THE DRAWINGS

FIG. 1 is a plan view of the atraumatic suction catheter of the present invention;

FIG. 2 is sectional view of FIG. 1 taken at line 2;

FIG. 3 is a sectional view similar to FIG. 2, showing the detail of one of the openings near the distal tip of the catheter;

FIG. 4 is a cross-sectional view of FIG. 2 taken along lines 4-4;

FIG. 5A is a perspective view of the distal tip of FIGS. 1-3;

FIG. 5B is a side view of the distal tip of FIGS. 1-3;

FIG. 5C is a plan view of the distal tip of FIGS. 1-3;

FIG. 6A is a perspective view of the tip of an embodiment of another atraumatic suction catheter;

FIG. 6B is a side view of FIG. 6A;

FIG. 6C is a plan view of the distal tip of FIGS. 6A and 6B;

FIG. 7A is a perspective view of the tip of yet another embodiment of an atraumatic suction catheter;

FIG. 7B is a side view of the tip of FIG. 7A;

FIG. 7C is a plan view of the tip of FIGS. 7A and 7B;

FIG. 8A is a perspective view of the tip of still yet another embodiment of an atraumatic suction catheter;

FIG. 8B is a side view of the tip of FIG. 8A;

FIG. 8C is a plan view of the tip of FIGS. 8A and 8B;

FIG. 9 is a Table illustrating Impact Force vs Speed; and

FIG. 10 is a Table slowing Impact Ratio vs Speed.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

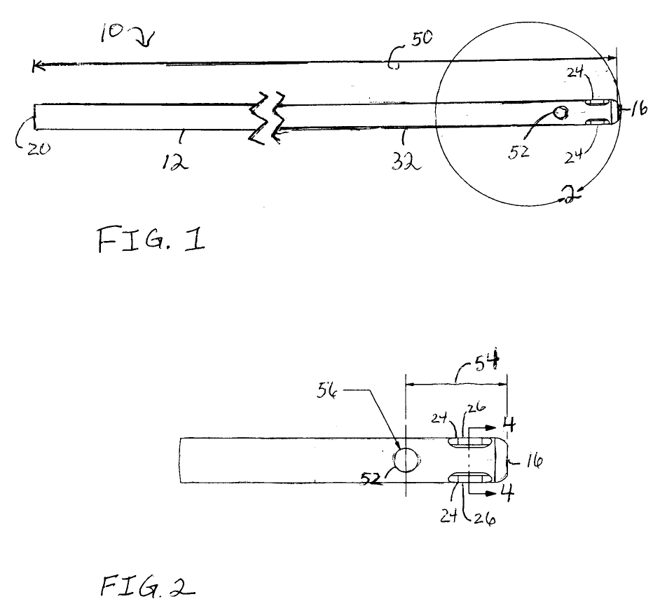

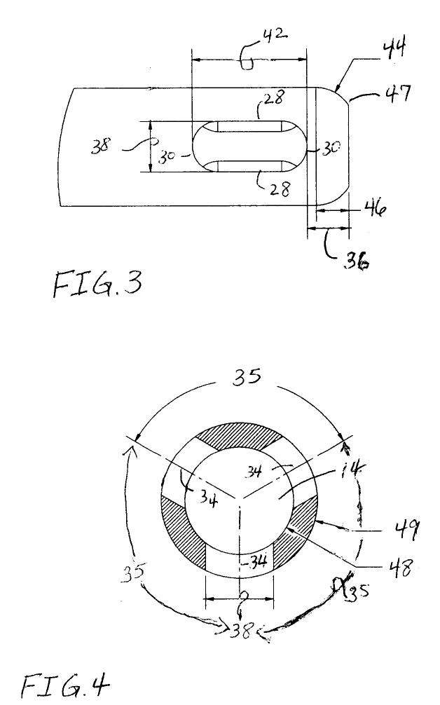

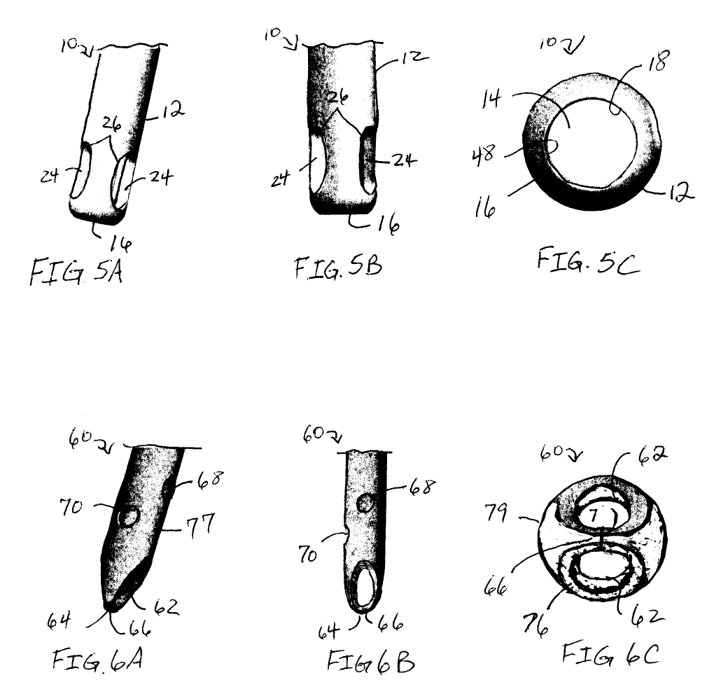

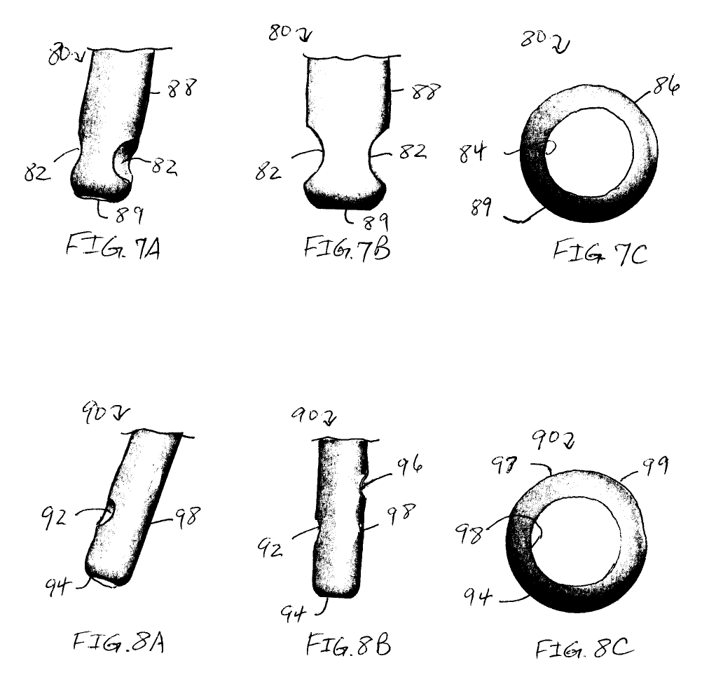

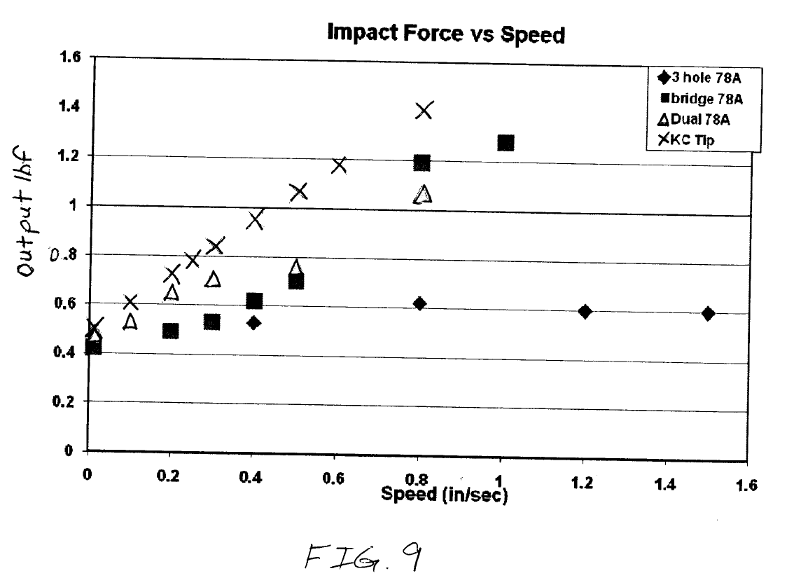

Turning now to the drawings, as illustrated in FIGS. 1-4 and 5A-C, an atraumatic suction catheter 10 is provided. The suction catheter 10 includes an elongated body 12 having an opening or lumen 14 provided therethrough. A distal end or tip 16 is beveled or formed to have a continuous radius, and has an opening 18 formed therein in communication with the lumen 14. An opposite, proximal end 20 has an opening (not shown) formed therein which is also in communication with the lumen 14.

Three openings (collectively “24”) are provided near the distal tip 14. Each opening 24 is desirably elongated and a perimeter 26 of each opening 24 may desirably be formed to have parallel opposing sides 28. The perimeter 26 of each opening 24 may also be formed to have opposing rounded ends 30 which are desirably, but not by way of limitation, U-shaped. Each of the three openings 24 is desirably equally-sized and equally spaced about an outer circumference 32 of the catheter 10. That is, a center 34 of each opening 24 is spaced to be about 120 degree angle 35 from each adjacent center 34 of each opening 20. In another alternative (not shown), the openings 24 are each formed of the same size and positioned in the same position, and are of about the same length and about the same width, but are instead oval or elliptical-shaped.

Each opening 24 is positioned near but spaced (relative to its most distal portion) a distance 36 from the distal tip 14. Each opening 24 may be spaced a distance 36 in a range of about 0.085 to about 0.045 inch from the distal tip 14. Desirably, each opening 24 may be spaced a distance 36 of in a range of about 0.075 to about 0.055 inch from the distal tip 14. More desirably, each opening 24 may be spaced a distance 36 in a range of about 0.070 to about 0.060 inch from the distal tip 14, with a most desirable distance between the opening 24 and the distal tip 14 of about 0.065 inch.

Each opening 24 may have a width 38. Each opening 24 may have a width 38 between its elongated sides 28 in a range of about 0.110 to about 0.050 inch. Desirably, the width 38 between the elongated sides 28 may be in a range of about 100 to about 0.060 inch. More desirably, the width 38 between the elongated sides 28 may be in a range of about 0.090 to about 0.070 inch, with a most desirably width 38 of about 0.080 inch.

Each opening 24 also has a length 42. Each opening 24 may have a length 42 of about 0.200 to about 0.160 inch. Desirably, each opening 24 may have a length 42 of about 0.190 to about 0.170 inch. More desirably, each opening 24 may have a length 42 of about 0.185 to about 0.175 inch, and most desirably a length 42 of about 0.180 inch.

The distal tip 14 is beveled. The curvature 44 of the distal tip 14 may have a radius in a range of about 0.085 to about 0.045 inch. The curvature 44 of the distal tip 14 desirably has a radius of about 0.075 to about 0.055 inch. The curvature 44 of the distal tip 14 more desirably has a radius of about 0.070 inch to about 0.060 inch, and most desirably a radius of about 0.065 inch.

The distance 46 between the beginning of the curvature 44 of the distal tip 14 and the most distal point of the distal tip 14 may be in a range of about 0.070 to about 0.030 inch. Desirably, the distance 46 may be in a range of about 0.060 to about 0.040 inch. More desirably, the distance 46 may be in a range of about 0.055 to about 0.045 inch, and most desirably the distance 46 is about 0.051 inch.

A perimeter 47 of the opening 18 may have a diameter within a range of about 0.110 to about 0.150 inch. The diameter 47 desirably may be formed within a range of about 0.120 to about 0.140 inch. The diameter 47 more desirably may be formed within a range of about 0.132 to about 0.123 inch, and most desirably the diameter 48 is about 0.128 inch.

The body 12 of the catheter has an inner diameter 48. The inner diameter 48 may be formed within a range of about 0.110 to about 0.150 inch. The inner diameter 48 desirably may be formed within a range of about 0.120 to about 0.140 inch. The inner diameter 48 more desirably may be formed within a range of about 0.132 to about 0.123 inch, and most desirably the inner diameter 48 is about 0.128 inch.

The body 12 of the catheter has an outer diameter 49. The outer diameter 49 may be formed within a range of about 0.165 to about 0.205 inch. The outer diameter 49 desirably may be formed within a range of about 0.175 to about 0.195 inch. The outer diameter 49 more desirably may be formed within a range of about 0.180 to about 0.190 inch, and most desirably the outer diameter 49 is about 0.188 inch.

The inner and outer diameters will vary, however, according to what diameter of catheter size is chosen by a user as appropriate to use. Common catheter sizes range for example, but not by way of limitation, from a 5 French to an 18 French, although the present invention and all alternative designs described herein are 14 French.

The catheter 10 also includes a length 50. The length 50 may be in a range of about 25 to about 10 inches. Desirably, the length 50 may be in a range of about 23 to about 18 inches. More desirably, the length 50 may be in a range of about 22 to about 19 inches, and most desirably about 20.87 inches. It will be understood, however, that other shorter or longer lengths may be utilized.

A round aperture 52 is provided a distance proximally relative to the three openings 24. The round aperture 52 is desirably, but not by way of limitation, spaced an equal distance between two of the three openings 24, and a distance proximally as well.

The round aperture 52 may be positioned a distance 54 proximally from the distal tip 16, and the distance may be in a range of about 0.350 to about 0.500 inch from the distal tip 14. Desirably, the distance 54 of the round aperture 52 is about 0.400 to about 0.475 inch from the distal tip 14. More desirably, the distance 54 of the round aperture 52 is about 0.414 to about 0.441 inch from the distal tip 14, and most desirably the distance 54 of the round aperture 52 is about 0.421 inch from the distal tip 14.

A diameter 56 of the round aperture 52 may be about 0.080 to about 0.120 inch diameter. The diameter 56 of the round aperture 52 desirably may be about 0.090 to about 0.110 inch diameter. More desirably, the diameter 56 of the round aperture 52 may be about 0.095 to about 0.105 inch diameter, and most desirably, the diameter is about 0.100 inch diameter.

The suction catheter 10 is desirably made from one or more polymers. More desirably, the suction catheter 10 is constructed from a phthalate-free polyvinylchloride (PVC).

The present design, as shown and described herein in FIGS. 1-4 and 5A-5C (the present invention is referred to as the “Three Distal Hole” suction catheter 10 design even though there is a more proximally positioned fourth aperture as well as a distal-most opening in the distal tip), was tested against other suction catheter designs. That is, the present design was evaluated against the following:

Bridge tip design: a suction catheter 60 illustrated in FIGS. 6A-6C, which is made from the same material as the present invention, and which has the same construction, except that the suction catheter has two large oval openings (collectively “62”) and a bridge 64 providing a distal tip 66. The bridge tip design also has two additional apertures 68, 70 which are set above the two oval openings 62 at different distances from one another and the distal tip 66. The shape and position of the oval openings 62 cause the distal tip 66 to tapered toward the bridge.

Specifically, the bridge tip catheter 60 may have a total length (not shown) of about 20.87 inches. Each oval opening 62 at the bridge 64 may have a length of about 0.236 inch. The bridge 64 between the two oval openings 62 may have a width 74 of about 0.021 inch. The perimeter 76 of each oval side opening 62 is desirably positioned at about a 21 degree angle relative to the outer non-tapered perimeter 77 of the bridge tip suction catheter 60. The bridge tip catheter has two additional round openings 68, 70, each desirably having a radius of about 0.08 inch. The proximal opening 68 is desirably positioned 0.50 inch from the distal tip 64, and it is positioned directly below one of the oval side openings and axially aligned with it. The distal opening 70 is positioned about 90 degrees away from the proximal opening 68, and it is positioned about 0.35 inch from the distal tip 64. The inner diameter (not shown) of the catheter is desirably about 0.128 inch; the outer diameter 79 of the catheter is desirably about 0.184 inch.

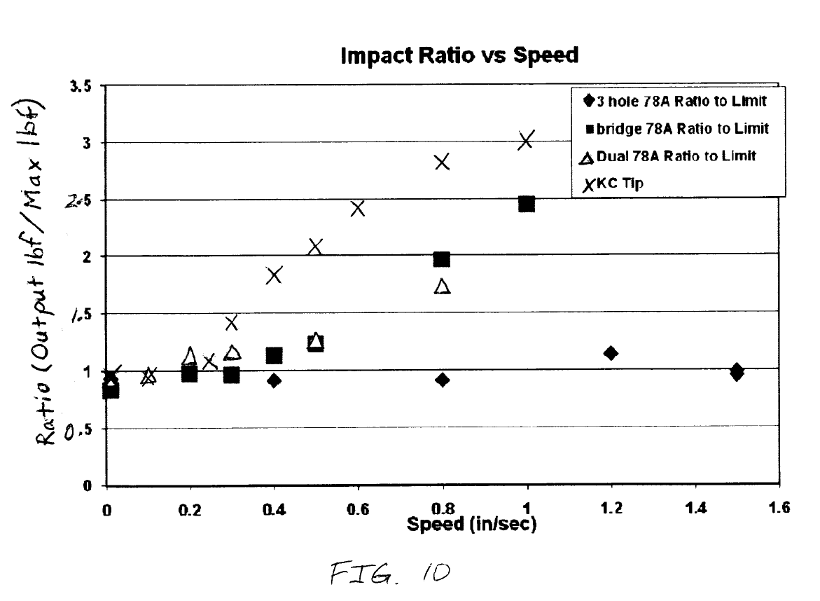

Dual side hole design: a suction catheter 80 shown in FIGS. 7A-7C made from the same material as the present invention, and which has the same construction, but having two openings (collectively “82”) placed directly across from one another on either side of the catheter 80. The catheter 80 desirably has a length (not shown) of about 20.5 inches. The inner diameter 84 of the catheter is desirably about 0.128 inch; the outer diameter 86 of the catheter is desirably about 0.184 inch. The beveled end of the distal tip 89 may desirably be identical to the beveled end 16 of the Three Distal Hole design suction catheter 10. The holes 82 are positioned 180 degrees opposite each other on the perimeter 88 of the suction catheter 80, and each desirably has a radius of about 0.10 inch. Each hole 82 is positioned 0.25 inch from the distal tip, Off-set hole design: a suction catheter 90 illustrated in FIGS. 8A-8C made from the same material as the present invention, and which has the same construction and generally identical to the Dual Side Hole design, except that the suction catheter 90 has a distal hole 92 desirably positioned about 0.25 inch from the distal tip 94, and a second, proximal hole 96 which is positioned about 180 degrees away from the distal hole 92 on an opposite side of a perimeter 97 of the Off-Set Hole suction catheter 90, and which is spaced about 1.75 inch from the distal tip 94. The inner diameter 98 of the catheter is desirably about 0.128 inch; the outer diameter 99 of the catheter is desirably about 0.184 inch.

Finite element analysis (FEA) was conducted for each of the above-referenced designs.

Each catheter was held about 0.797 inch from the distal tip, and the tip of each catheter was positioned about 0.797 inch above a simulated tracheal model. Each catheter had a 0.5 force applied axially with a uniform distribution onto the simulated tracheal model (mesh). Certain features of the tracheal tissue, 60A PVC and 78A PVC are outlined below. Tracheal properties are from Strength of Biological Material by Hiroshi Yamada, published by Robert E. Krieger Publishing Company, Huntington, N.Y. 1973, p. 141-142. PVC properties referenced herein are from www.Matweb.com (which provides material property data and information).

Material Characteristics

| | Tracheal Tissue | 60A PVC | 78A PVC |
|---|---|---|---|
| Tensile Strength | 341.36 psi | 1600 psi | 2300 psi |
| Elastic Modulus | 2702.34 psi | 550 psi | 1200 psi |
| Poisson's Ratio | — | 0.3 | 0.3 |
| Yield Strength | — | 1600 psi | 2300 psi |

A mesh was utilized to model tracheal tissue, as follows:

| Mesh Type: | Solid Mesh/Standard: | |
|---|---|---|
| Jacobean Check | 16 | Points |
| Element Size | 0.04549 | inch |
| Tolerance | 0.0022745 | inch |

| | |
|---|---|
| Number of Elements | 71177 |
| Number of Nodes | 111786 |

Restraints were located on 2 fixed faces of the tracheal model. Force applied was 0.5 lbs applied along an axial alignment with respect to each distal tip of each design with uniform distribution. Contact set: touching faces, but no penetration between the selected tracheal model and the selected design tip. The program used was CosmosWorks 2008 which is associated with SolidWorks 2008 SP3.1.

TABLE 1

| | Tip Stress (psi) | Contact Stress (psi) |
|---|---|---|
| Three Distal Hole - 78A | 125 | 65 |
| Bridge Tip - 78A | 320 | 280 |
| Bridge Tip - 60A | 210 | 137 |
| Dual Side Holes - 60A | 165 | 120 |
| Off-Set Holes - 78A | 178 | 134 |

The term “tip stress” (measured in psi) as used in herein and documented in Table 1 means the stress distributed within the tip. The term “contact stress” (measured in psi) as used herein and documented in Table 1 means the stress distributed to the tracheal tissue.

Based on the results shown in Table 1, the three distal hole and the dual side holes designs performed comparably or slightly better than the off-set hole design. The decreased contact area of the bridge tip resulted in considerably larger contact stress. To refine the analysis, forces that might be expected due to the advancement of a distal end of a catheter to a carina (the downward and backward projection of the last tracheal cartilage, which forms a ridge that separates the opening of the right and left main stem bronchi), as well as the effect of catheter durometer (i.e., 78A, 72A or 60A) determined. Therefore, the FEA simulations were again tested on each design, using 0.2 lbs , 0.5 lbs and 1 lb force, and the resulted are noted herein in Table 2.

TABLE 2

| | Contact Stress (psi) |
|---|---|
| Three Distal Hole - 78A (0.2 lb insertion force) | 49 |
| Three Distal Hole - 78A (0.5 lb insertion force) | 110 |
| Three Distal Hole - 78A (1.0 lb insertion force) | 175 |
| Three Distal Hole - 60A (0.2 lb insertion force) | 34 |
| Three Distal Hole - 60A (0.5 lb insertion force) | 99 |
| Three Distal Hole - 60A (1.0 lb insertion force) | 171 |
| Bridge Tip - 78A (0.2 lb insertion force) | 128 |
| Bridge Tip - 78A (0.5 lb insertion force) | 280 |
| Bridge Tip - 78A (1.0 lb insertion force) | 330 |
| Bridge Tip - 60A (0.2 lb insertion force) | 117 |
| Bridge Tip - 60A (0.5 lb insertion force) | 156 |
| Bridge Tip - 60A (1.0 lb insertion force) | 278 |
| Dual Side Holes - 78A (0.2 lb insertion force) | 49 |
| Dual Side Holes - 78A (0.5 lb insertion force) | 135 |
| Dual Side Holes - 78A (1.0 lb insertion force) | 198 |
| Dual Side Holes - 60A (0.2 lb insertion force) | 49 |
| Dual Side Holes - 60A (0.5 lb insertion force) | 120 |
| Dual Side Holes - 60A (1.0 lb insertion force) | 148 |
| Off-Set Holes - 78A (0.2 lb insertion force) | 50 |
| Off-Set Holes - 78A (0.5 lb insertion force) | 134 |
| Off-Set Holes - 78A (1.0 lb insertion force) | 210 |

The results from Table 2 illustrate that the three distal side holes design performs similarly to the off-set holes design at low insertion forces (0.2 lbs), but had less contact stress at higher insertion forces, which appears to be due to the shock-absorbing nature of the design. Notably, the variation in durometer did not impact the results of the three hold design compared to the other designs, the improvement in contact force appeared to be obtained by geometry alone.

The dual side hole design performed similarly when compared to the off-set hole design and the three distal hole design. The concern with this catheter design, however, was that at a lower durometer, the catheter collapses to an extent which may block or significantly effecting suctioning.

The bridge tip design, at both durometers, performed consistently worse than the other designs. The data suggests that the decreased contact area presented by the bridge tip design concentrates the applied force.

Significantly, it was determined that by adjusting the geometry of the three distal hole design, a reduction in the applied force was obtained. Other manufacturers have attempted to reduce applied force by adjusting the durometer of a suction catheter distal tip, to make it softer. In doing so, however, the softer distal tip often collapses and significantly effects suctioning efficiency. The present avoids this problem.

Suction efficiency testing was performed on the designs. The testing was based on Shah, Samir, Kung, Kevin, et al., An In Vitro Evaluation of the Effectiveness of Endotracheal Suction Catheters, Chest 2005;128:3699-3705.

An A-Vac Industries Vacuum Pump, DV-4E 4CFM, a Control Air Inc. Pressure Regulator, 0-15 psi range, a vacuum chamber with pressure gauge, an Ohaus Adventurer Pro Scale Model AV81011, I-019, a Brookfield Digital Viscometer, LVTDV-II, and a Polyox Water Soluble Resin Coagulant, from Dow Chemical Company, Cary, N.C., were utilized.

The vacuum pump was connected to the pressure regulator. The pressure regulator was connected to the vacuum chamber. The catheter design being tested was connected to the vacuum chamber. All connections were evaluated to ensure they were air tight and UV cure Loctite was added to appropriate connection on the vacuum chamber to ensure sealing. The Polyox coagulant (comprising polyetheyine oxide) was mixed with water at concentrations of 0.5%, 1.5% and 3% to simulate mucous of different viscosities.

To achieve concentrations of 0.5%, 1.5% and 3% polyethylene oxide to water, appropriate amounts of polyethylene oxide and water were weighed in separate beakers and set aside. Each beaker containing water was placed in a water bath or a heating plate until the water temperature was 95 degrees Celsius. The Polyox powder was added to the water and the combined solution was stirred continuous, then removed from the heat source. The mixtures sat for two hours, and were stirred periodically.

The test was conducted by attaching the catheter being tested to the vacuum chamber and ensuring that all connections were air tight. Each catheter tested was supported and the distal end of the catheter was inserted until it was submerged into the polyethylene oxide water solution. The vacuum was turned on and the pressure regulator was used until the pressure gauge read the appropriate value inside the vacuum chamber. The scale was zeroed. Suction was applied to the catheter for five (5) seconds for each coagulant mixture, at 120 mm Hg and 300 mm Hg. The value on the scale was recorded; the amount of mucous suctioned was reported in grams. The catheter was inserted into water, and suction was applied until the catheter was rinsed clean. Five (5) of each of the four different suction catheter designs were utilized in the test. The tip of each catheter design was completely submerged in the solution, however, the upper aperture of the three distal hole design was not submerged. The process was repeated once with five (5) times per design. The results (secretions suction in grams) are provided herein in Table 3.

TABLE 3

| | 120 mm Hg | | | 300 mm Hg | | |
|---|---|---|---|---|---|---|
| Design | 0.5% | 1.5% | 3.0% | 0.5% | 1.5% | 3.0% |
| 3 Distal | | | | | | |
| Hole | | | | | | |
| Average | 5.4 | 3.22 | 0.68 | 11.6 | 6.08 | 2.38 |
| St. Dev. | 1.30 | 0.55 | 0.43 | 1.64 | 0.29 | 1.51 |
| Maximum | 6.5 | 4.2 | 1.2 | 13.7 | 6.3 | 4.7 |
| Minimum | 3.6 | 2.9 | 0.2 | 9.4 | 5.6 | 0.5 |
| Dual Side | | | | | | |
| Hole | | | | | | |
| Average | 5.98 | 2.36 | 0.36 | 11.36 | 4.16 | 1.12 |
| St. Dev. | 0.57 | 1.26 | 0.11 | 0.91 | 2.02 | 0.26 |
| Maximum | 6.7 | 4.0 | 0.5 | 12.6 | 6.2 | 1.5 |
| Minimum | 5.4 | 1.2 | 0.2 | 10.2 | 2.2 | 0.8 |
| Bridge Tip | | | | | | |
| Design | | | | | | |
| Average | 5.46 | 2.16 | 0.22 | 12.04 | 5.12 | 1.7 |
| St. Dev | 0.84 | 1.05 | 0.16 | 1.11 | 0.95 | 0.72 |
| Maximum | 6.8 | 3.1 | 0.4 | 13.4 | 6.0 | 2.4 |
| Minimum | 4.8 | 0.5 | 0.1 | 10.3 | 3.5 | 0.5 |
| Off-set | | | | | | |
| Holes | | | | | | |
| Average | 5.82 | 2.12 | 0.3 | 10.5 | 3.24 | 0.22 |
| St. Dev | 0.97 | 1.03 | 0.12 | 0.96 | 0.15 | 0.08 |
| Maximum | 6.7 | 3.9 | 0.5 | 11.8 | 3.4 | 0.7 |
| Minimum | 4.2 | 1.7 | 0.2 | 9.1 | 1.0 | 0.1 |

At low viscosity, all concepts performed equivalently. As the viscosity of the viscous solutions increased, suction efficiency decreased for all designs. The three distal hole design of the present invention did have improved suction efficiency as compared to the other concepts. This was also evident as the suction pressure was increased from 120 mm Hg to 300 mm Hg.

Therefore, the average amount of 0.5% simulated mucous suction in 5 seconds at 120 mm HG was 5.4 grams. The average amount of 1.5% simulated mucous suction in 5 seconds at 120 mm HG was 3.22 grams. The average amount of 3.0% simulated mucous suction in 5 seconds at 120 mm HG was 0.68 grams. The average amount of 0.5% simulated mucous suction in 5 seconds at 300 mm HG was 11.6 grams. The average amount of 1.5% simulated mucous suction in 5 seconds at 300 mm HG was 6.08 grams. The average amount of 3.0% simulated mucous suction in 5 seconds at 300 mm HG was 2.38 grams.

The various designs were also subjected to impact force testing. The test was conducted to evaluate the force at impact, independent from the surface area but dependent on the rate of insertion.

The equipment used included a Loyd Tensile Tester, a Nexygen software package, a force gauge, and each suction catheter design. Grips were connected to the upper load cell and secured with the load cell pin. Each catheter tip was positioned so that the distal tip extended downward about 0.797 inch from the grip. The force gauge was affixed directly under the load cell and the platform was aligned directly under the distal tip, which was positioned about 0.797 inch thereabove. The display screen of the force gauge was adjusted to be visible. The Nexygen file was set up with the following parameters: Test: compression; speed: I inch/second; limit: stop at load of 1.0 lbf (pound/force).

In running the test, the height of the load cell was set 2 inches above the base of the force gauge (a calibrated rule was used to determine height). The force value and height of the Lloyd tester was zeroed from the side panel of the machine. The Nexygen interface was activated by pressing “play”, and the test was allowed to complete (the program prompts the user at the conclusion of the test). The top fixture was returned to the zero position with the arrows on the side of the Lloyd Tester. The information on the force gauge (maximum force) was recorded. The steps were repeated for each design sample, varying the input force and speed as noted herein.

Each suction catheter design was tested at 0.5 inch/s and the impact force was measured with an input force of 0.5 lbs, 0.1 lbf, 0.5 lbf and 1.0 lbf. Six (6) Three-Distal Hole tip suction catheter tips were used for each of the tests at 0.1 lbf, 0.5 lbf and 1.0 lbf. Seven (7) of the Bridge Tip suction catheter tips were used for each of the tests at 0.1 lbf, 0.5 lbf and 1.0 lbf. Six (6) Dual Hole tip suction catheter tips were used for each of the tests at 0.1 lbf, 0.5 lbf and 1.0 lbf. Ten of the Off-Set Hole tip suction catheter tips were used for each of the tests at 0.1 lbf, 0.5 lbf and 1.0 lbf. The results are illustrated in FIG. 9.

FIG. 9 shows the correlation between impact force and the rate (speed) of insertion at 0.5 lbs of insertion force. At slow insertion rates, as illustrated in FIG. 9, all suction catheter designs performed similarly. At higher insertion rates, however, the three distal hole design of the present invention had the lowest impact force, and its impact force appears to be independent of the rate of insertion. This is because the design has a lower buckling threshold, and once the catheter buckles, the output force does not change. Therefore, this design displays significant “shock absorption” characteristics. That is, at a speed of 0.4 in/sec, the output lbf of the three hole design was less than about 0.6 lbf. Further, as the speed increased to 0.8 in/sec, the output of of the three hole design was less than about 7 lbf, and less than about 0.65 lbf. At 1.2 in/sec, the output lbf of the three hole design was about 0.6 lbf. And, at about 1.55 in/sec. the output lbf of the three hole design was about 0.6 lbf.

The impact ratio (output lbs force vs. maximum lbs force) was also calculated to present the data independently of the insertion force, as illustrated in FIG. 10. The impact ratio also demonstrates that the three distal hole design operates independently of the insertion speed, as shown in FIG. 10, and has a greatly reduced impact ratio as compared to the other designs. At an insertion speed of about 0.4 in/sec, the impact ratio for the three hole design was less than about 1. At an insertion speed of about 0.8 in/sec, the impact ratio was less than about 1. At an insertion speed of about 1.2 in/sec, the impact ratio was less than about 1.25. At an insertion speed of about 1.55, the impact ratio was about 1.

Temperature for all tests described herein was about 72 degrees F.±2 degrees. Relative humidity was about 45 percent, ±5 percent.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed is:

1. A suction catheter, comprising:
a tube-shaped body having a lumen formed therethrough, a beveled distal tip having an opening therein in communication with the lumen, a proximal end having an opening in communication with the lumen and adapted to be coupled to a suction source;
wherein the distal tip has three equally-spaced apertures positioned near the distal tip, each of the three apertures having a perimeter defining a pair of parallel elongated sides and opposing U-shaped ends,
wherein when the distal tip of the suction catheter is moved at a speed of about 0.4 inches/second, it has an impact force of less than about 0.6 lbf.

2. The suction catheter of claim 2, wherein when the distal tip of the suction catheter is moved at a speed of about 0.8 inches/second, it has an impact force of less than 0.7 lbf.

3. The suction catheter of claim 3, wherein when the distal tip of the suction catheter is moved at a speed of about 1.2 inches/second, it has an impact force of about 0.6 lbf.

4. The suction catheter of claim 4, wherein when the distal tip of the suction catheter is moved at a speed of about 1.55 inches/second, it has an impact force of about 0.6 lbf.

5. A suction catheter, comprising:
a tube-shaped body having a lumen formed therethrough, a beveled distal tip having an opening therein in communication with the lumen, a proximal end having an opening in communication with the lumen and adapted to be coupled to a suction source;
wherein the distal tip has three equally-spaced apertures positioned near the distal tip, each of the three apertures having a perimeter defining a pair of parallel elongated sides and opposing U-shaped ends, wherein when the distal tip of the suction catheter is moved at a speed of about 0.4 inches/second, it has an impact ratio less than 1.

6. The suction catheter of claim 5, wherein when the distal tip of the suction catheter is moved at a speed of about 0.8 inches/second, it has an impact ratio less than 1.

7. The suction catheter of claim 6, wherein when the distal tip of the suction catheter is moved at a speed of about 1.2 inches/second, it has an impact ratio less than about 1.25.

8. The suction catheter of claim 7, wherein when the distal tip of the suction catheter is moved at a speed of about 1.55 inches/second, it has an impact ratio less than about 1.

9. A suction catheter, comprising:
a tube-shaped body having a lumen formed therethrough, a beveled distal tip having an opening therein in communication with the lumen, a proximal end having an opening in communication with the lumen and adapted to be coupled to a suction source;
three equally-spaced apertures positioned near the distal tip, each of the three apertures having a perimeter defining a pair of parallel elongated sides and opposing U-shaped ends; and
one round aperture spaced a distance proximally from the three apertures,

10. The suction catheter of claim 9, wherein the catheter has improved suctioning of viscous secretions at a vacuum of both 120 mm HG and 300 mm Hg, and wherein the viscous secretions comprise a mixture of 0.5 percent polyethylene oxide in water.

11. The suction catheter of claim 9, wherein the catheter has improved suctioning of viscous secretions at a vacuum of both 120 mm HG and 300 mm Hg, wherein the viscous secretions comprise a mixture of 1.5 percent polyethylene oxide in water.

12. The suction catheter of claim 9, wherein the catheter has improved suctioning of viscous secretions at a vacuum of both 120 mm HG and 300 mm Hg, wherein the viscous secretions comprise a mixture of 3 percent polyethylene oxide in water.
